(19) United States
(12) Patent Application Publication
(10) Pub. No.: US 2024/0215879 A1
(43) Pub. Date: Jul. 4, 2024

(54) TRANSPORT BAG FOR TRANSPORTING LIQUID SAMPLES AND METHOD FOR THE PRODUCTION THEREOF

(71) Applicant: Anton Debatin GmbH Werk Fuer Werbende Verpackung, Bruchsal (DE)

(72) Inventors: Thomas ROSE, Hann. Muenden (DE); Oliver TROEBER, Brettten (DE)

(73) Assignee: Anton Debatin GmbH Werk Fuer Werbende Verpackung, Bruchsal (DE)

(21) Appl. No.: 18/607,470

(22) Filed: Mar. 17, 2024

Related U.S. Application Data
(63) Continuation of application No. PCT/EP2022/077350, filed on Sep. 30, 2022.

(30) Foreign Application Priority Data
Oct. 7, 2021 (DE) 10 2021 125 990.8

(51) Int. Cl. A61B 5/15

(52) U.S. Cl. CPC A61B 5/150366; A61B 5/150274; A61B 5/150358

(57) ABSTRACT

In order to produce a transport bag for transporting liquid samples, in particular blood, urine, tissue samples, or the like, two film layers made of plastic are placed one on top of the other and joined to one another in such a manner that an interior space is formed between the film layers. Before the two film layers are placed one on top of the other, a liquid or viscous carrier material into which absorber particles are or will be mixed is applied to at least one of the film layers. Subsequently, the carrier material with the absorber particles is dried and/or cooled. Preferably, the carrier material is sprayed or spattered or printed or knife coated or brushed onto the film layer.

This nonprovisional application is a continuation of International Application No. PCT/EP2022/077350, which was filed on Sep. 30, 2022, and which claims priority to German Patent Application No. 10 2021 125 990.8, which was filed in Germany on Oct. 7, 2021, and which are both herein incorporated by reference.

### BACKGROUND OF THE INVENTION

### Field of the Invention

The invention relates to a transport bag for transporting liquid samples, in particular blood, urine, tissue samples, or the like, having a first film layer and a second film layer that are each made of plastic and are arranged one on top of the other and joined to one another, forming an interior space between the film layers, wherein an absorption device for accommodating liquid is arranged in the interior space, which absorption device has absorber particles that are arranged on and/or in a carrier material, wherein the carrier material is held on the inner side of at least one of the film layers facing the interior space.

### Description of the Background Art

In addition, the invention relates to a method for producing a corresponding transport bag, wherein two film layers made of plastic are placed one on top of the other and joined to one another in such a manner that an interior space is formed between the film layers.

A transport bag of the said type is described in DE 202012100517 U1, for example. When liquid samples are transported, there is a risk that a sample container accommodating the liquid sample either begins to leak or is damaged so that the sample inside the transport bag leaks out. In order to prevent the liquid sample from reaching the environment, it is known to wrap the sample container in an absorbent material, for example precut nonwoven fabric, before placing it in the transport bag. Alternatively or in addition thereto, an absorbent element, for example precut nonwoven fabric, can also be placed in the interior space of the transport bag, wherein additional particles of SAP (superabsorbent polymer) may be added. The prefabricated, absorbent element can be adhesive-bonded to the inner wall of at least one of the film layers. In all cases, the prefabrication, the attachment, or the insertion of the absorbent element is laborious and time-consuming, making the production of the transport bag cost-intensive.

### SUMMARY OF THE INVENTION

It is therefore an object of the invention to create a method for producing a corresponding transport bag that permits automated production of the transport bag. In addition, it is also an object of the invention to create a transport bag that can be produced in a simple manner.

The abovementioned object is attained according to an example of the invention in that a liquid or viscous carrier material into which absorber particles are or will be mixed is applied to at least one of the film layers before the two film layers are placed one on top of the other. The application of the carrier material can be accomplished by, for example, spraying, spattering, printing, knife coating, or brushing. Subsequently, the carrier material with the absorber particles contained therein is dried and/or cooled. The drying and/or the cooling can occur in the ambient air and/or by supplying, for example blowing, warm air for drying or cold air for cooling. Preferably, the drying and/or cooling takes place before the film layers are placed one on top of the other.

A curable plastic and/or adhesive can be used as the carrier material. This is associated with the advantage that the absorber particles can be mixed into and/or embedded in the still-liquid or viscous plastic and/or adhesive, whereupon the plastic and/or adhesive can be applied to at least one of the film layers and cures there, and consequently is joined to the corresponding film layer. Preferably, the absorber particles are already incorporated in the carrier material before it is applied to the film layer.

Examples of materials that can be used as carrier materials are polyethylene oxides, poly-N-vinylpyrrolidones, polyvinyl alcohols, and polyacrylamides, or a mixture of these materials.

Such absorber particles can be used that are formed at least predominantly of at least one superabsorbent polymer (SAP). A corresponding polymer is capable of soaking up a multiple of its own weight in polar liquids, in particular water or aqueous solutions.

The superabsorbent polymer can be, for example, a copolymer of acrylic acid or sodium acrylate on the one hand and acrylamide on the other hand.

The absorber particles can preferably be used as a granulate with a particle size in the range from 100 μm to 1000 μm.

The absorber particles can be present in a proportion of 20% mass to 80% mass of the total mass of carrier material and absorber particles. In particular, a proportion of 30% mass to 50% mass of the total mass of carrier material and absorber particles is provided for the absorber particles.

The carrier material can be permanently joined and, for example, fused or joined in a material-to-material manner, to the film layer holding it. Provision can also be made that the carrier material can adhere peelably to the film layer so that a separation of the carrier material from the film layer of the transport bag is possible even after use of the transport bag.

The carrier material may be designed in a strip shape, for example, and can be arranged in the end region of the transport bag that faces away from an insertion opening through which the interior space of the transport bag can be accessed. The bottom third or the bottom quarter of the length of the transport bag may be viewed as the end region.

At each of its side edges, the transport bag can be joined by means of a weld seam that joins the two film layers. Alternatively, provision can be made that the carrier material extends essentially parallel to and/or at a distance from at least one of the weld seams.

Two strip-shaped carrier materials of the type can be present, in each of which the absorber materials are mixed and/or embedded.

The two film layers can be made from a single piece of film that is folded over in such a manner that the two film layers that lie on top of one another are formed while producing a folded edge. Prior to the folding of the film layers, the carrier material, for example in the form of a curable plastic and/or adhesive, is applied in the said manner to the film. The absorber particles can have been mixed into the plastic and/or adhesive even before the application of the plastic and/or adhesive. Alternatively or in addition thereto, provision can be made that the absorber particles are applied to the plastic and/or adhesive and/or are embedded therein after the application of the plastic and/or adhesive to the film.

After the carrier material or the plastic and/or adhesive is cooled and/or dried or has at least begun to dry, the film is folded or folded over, forming the two film layers that lie on top of one another, and the film layers are joined to one another by forming two weld seams arranged at a distance. The access opening can be provided between the film layers. Alternatively, it is possible to slit one of the film layers, wherein the slit thus produced forms the access opening.

A safety closure, such as is customary with safety transport bags, can be formed in or on the access opening. A corresponding safety closure may be designed such that it is recognizable from the outside when an attempt has been made after sealing of the bag to open it, for example through the action of heat, cold, or chemicals.

Also, a transport bag is provided, wherein an absorption device for accommodating liquid has absorber particles that are arranged on and/or in a carrier material, wherein the carrier material is held on the inner side of at least one of the film layers facing the interior space. As the carrier material, a cured plastic and/or adhesive can be provided that can be applied together with the absorber particles to the film in the said manner and, for example, can be sprayed or printed on. Embodiments and improvements of the transport bag can be derived from the above explanations regarding the method.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a front view of a transport bag according to an example of the invention for transporting liquid samples, and

FIG. 2 is a cross-section along II-II in FIG. 1.

### DETAILED DESCRIPTION

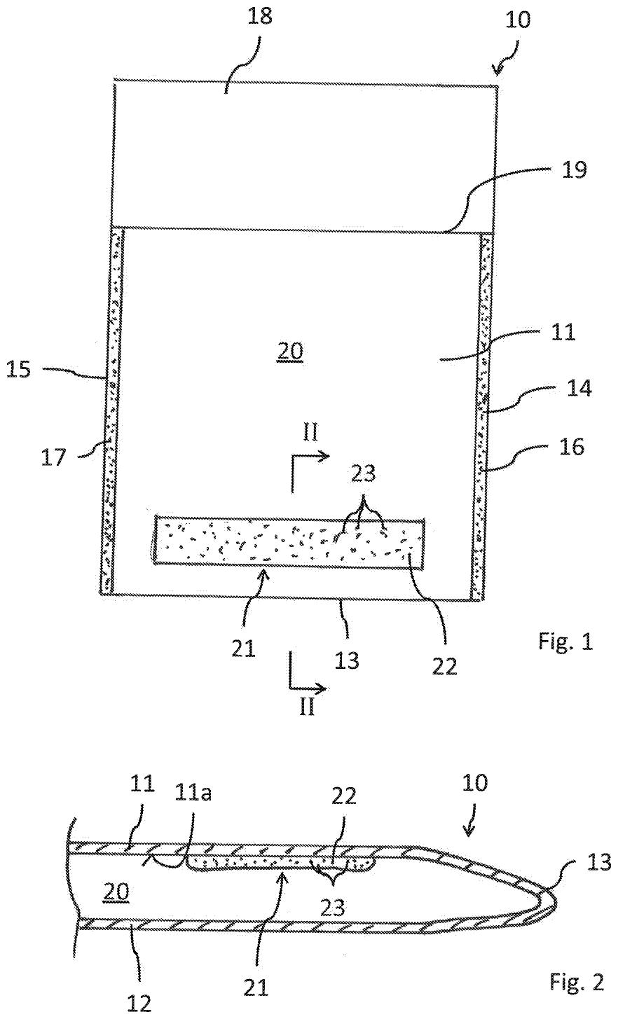

A transport bag 10 made of plastic for transporting liquid samples that is shown in FIGS. 1 and 2 has a first film layer 11 and a second film layer 12, which are arranged to lie stacked on top of one another. The two film layers 11 and 12 preferably are formed by the means that a plastic film is folded over and superposed while forming a bottom folded edge 13.

According to FIG. 1, the bottom, second film layer 12 projects past the top, first film layer 11 on the upper side of the transport bag 10 facing away from the folded edge 13, forming a flap 18.

The two film layers 11 and 12 are joined to one another by lateral weld seams 16 and 17 at their side edges 14 and 15 in such a manner that an interior space 20 is formed between the two film layers 11 and 12, which space is only accessible through an access opening 19 formed between the two film layers 11 and 12 in the region of the flap 18. The access opening 19 can be sealed by means of a closure, which can also be, for example, a so-called tamper-evident closure or a safety closure, which indicates later manipulation of the sealed transport bag 10.

Arranged in the interior space 20 of the transport bag 10 is an absorption device 21, by which means a liquid located in the interior space 20 can be soaked up and absorbed. The absorption device 21 includes a carrier material 22 in the form of a curable or cured plastic and/or adhesive, on and/or in which absorber particles 23 are held. The absorber particles 23 can be mixed into the carrier material 22 and/or spread thereon, for example.

In the example shown, the absorption device 21 can be arranged on the inner side 11a of the top first film layer 11 that faces the interior space 20, and is held on the first film layer 11. In this case, the absorption device 21 can be adhesive-bonded or joined in a material-to-material manner to the first film layer 11, wherein, if applicable, the absorption device 21 can be adhered to the first film layer 11 merely in a detachable manner and removed again later.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for producing a transport bag for transporting liquid samples, the method comprising:
placing two film layers, which are made of plastic, one on top of the other and fused to one another thereby forming a weld seam such that an interior space is formed between the two film layers;
applying a liquid or viscous carrier material, into which absorber particles are or will be mixed, to at least one of the two film layers before the two film layers are placed one on top of the other; and
drying and/or cooling the carrier material with the absorber particles after the carrier material is applied,
wherein the carrier material is sprayed or spattered or printed or knife coated or brushed on such that the carrier material is arranged at a distance from the weld seam.

2. The method according to claim 1, wherein the carrier material is applied to the film layer such that it adheres peelably to the film layer.

3. The method according to claim 1, wherein the carrier material is arranged in an end region of the transport bag that faces away from an insertion opening through which the interior space of the transport bag is accessed.

4. The method according to claim 1, wherein the carrier material comprises a curable plastic and/or an adhesive.

5. The method according to claim 1, wherein the absorber particles are formed at least predominantly of at least one superabsorbent polymer.

6. The method according to claim 1, wherein the absorber particles are provided in a proportion of ≥30% by mass in the carrier material.

7. A transport bag for transporting liquid samples, the transport bag comprising:
a first film layer;
a second film layer, the first film layer and the second film layer each made of a plastic and are arranged one on top of the other to form an interior space between the first and second film layers and are fused to one another thereby forming a weld seam; and
an absorption device arranged in the interior space, the absorption device accommodating a liquid that is held on an inner side of at least one of the first or second film layers facing the interior space,
wherein the absorption device has absorber particles that are arranged on and/or in a carrier material,
wherein the carrier material is a cured plastic and/or adhesive that is sprayed or spattered or printed or knife coated or brushed onto the at least one film layer,
wherein the absorber particles are mixed and/or embedded, and
wherein the carrier material is arranged at a distance from the weld seam.

8. The transport bag according to claim 7, wherein the carrier material adheres peelably to the film layer.

9. The transport bag according to claim 7, wherein the carrier material is arranged in an end region of the transport bag that faces away from an insertion opening through which the interior space of the transport bag is accessed.

10. The transport bag according to claim 7, wherein the absorber particles are formed predominantly of at least one superabsorbent polymer.

11. The transport bag according to claim 7, wherein the absorber particles are present in a proportion of ≥30% by mass in the carrier material.

12. The method according to claim 1, wherein the liquid samples include blood, urine, or tissue samples.
